(19) United States
(12) Patent Application Publication
(10) Pub. No.: US 2016/0376621 A1
(43) Pub. Date: Dec. 29, 2016

(54) FED BATCH PROCESS FOR BIOCHEMICAL CONVERSION OF LIGNOCELLULOSIC BIOMASS TO ETHANOL

(71) Applicant: GreenField Specialty Alcohols Inc., Toronto (CA)

(72) Inventors: Robert Ashley Cooper BENSON, North Bay (CA); Regis-Olivier BENECH, Chatham (CA)

(21) Appl. No.: 15/259,738

(22) Filed: Sep. 8, 2016

Related U.S. Application Data
(63) Continuation-in-part of application No. 12/751,459, filed on Mar. 31, 2010, now abandoned.
(60) Provisional application No. 61/166,490, filed on Apr. 3, 2009.
(60) Provisional application No. 61/169,107, filed on Apr. 14, 2009.

(51) Int. Cl. C12P 19/14; C12P 19/02

(52) U.S. Cl. CPC C12P 19/14; C12P 19/02; C12P 2201/00

(57) ABSTRACT

A method for optimization of a fed batch hydrolysis process wherein the hydrolysis time is minimized by controlling the feed addition volume and/or batch addition frequency of the prehydrolysate and optionally also the enzyme feed. The increase over time in hydrolysate consistency and volume and/or concentration of sugars released in the reactor, so that the enzymatic hydrolysis is controlled, significantly reduces the impact of cellulase feedback inhibition, especially for enzyme contents lower than 1%. The overall time to reach conversion of the total prehydrolysate feed is reduced significantly where the batch addition frequency is equal to one batch each time 70% to 90%, preferably 80%, conversion of the previous batch is reached in the reaction mixture. At an enzyme load of 0.3% in the reaction mixture, the optimum frequency each time 80% conversion was reached was found to be one batch every 80 to 105 minutes.

### CROSS REFERENCE TO RELATED APPLICATIONS

This application is a Continuation In Part Application of U.S. application Ser. No. 12/751,459, filed Mar. 31, 2010 and entitled Fed Batch Process for Biochemical Conversion of Lignocellulosic Biomass to Ethanol, which Application claims the benefit of priority of U.S. Provisional Patent Application No. 61/166,490 filed Apr. 3, 2009, and of U.S. Provisional Patent Application No. 61/169,107 filed Apr. 14, 2009, all of which which are hereby incorporated by reference.

### FIELD OF THE INVENTION

The present invention generally relates to the production of ethanol from biomass and in particular to a fed batch process for enzymatic hydrolysis of lignocellulosic biomass.

### BACKGROUND OF THE INVENTION

The importance of ethanol as a clean transportation fuel has increased with the anticipated shortage of fossil fuel reserves and with increased air pollution.

Ethanol is regarded as a more environmentally friendly fuel than gasoline because it adds less net carbon dioxide to the atmosphere. This is the main reason for significant research into economically viable ways of producing ethanol from renewable raw materials.

Fuel ethanol is distilled and dehydrated to create a high-octane, water-free alcohol. Ethanol is blended with gasoline to produce a fuel which has environmental advantages when compared to gasoline alone, and can be used in gasoline-powered vehicles manufactured since the 1980's. Most gasoline-powered vehicles can run on a blend consisting of gasoline and up to 10 percent ethanol, known as “E-10”.

Ethanol can be produced in several different ways. For example, ethanol can be synthesized from gasified carbon-containing feedstock. More commonly it is produced by the fermentation of sugar from starchy plants such as corn or wheat or sugar or from sugar cane or sugar beets.

In North America the feedstock is primarily corn, while in Brazil sugar cane is used. The use of potential food or feed plants to produce ethanol is considered as disadvantageous due to the limited availability of such feedstock and the limited area of suitable agricultural land.

An alternative to food or feed plants is lignocellulosic biomass. Biomass is widely available and contains a high proportion of cellulose, hemicellulose and lignin. The four main categories of biomass are: (1) wood residues (including sawmill and paper mill discards), (2) municipal paper waste, (3) agricultural residues (including corn stover and corn cobs and sugarcane bagasse), and (4) dedicated energy crops (which are mostly composed of fast growing tall, woody grasses such as switch grass and Miscanthus).

Lignocellulosic biomass is composed of three primary polymers that make up plant cell walls: Cellulose, a polymer of D-glucose; hemicellulose that contains two different polymers i.e. xylan, a polymer of xylose and glucomannan, a polymer of glucose and mannose; and lignin, a polymer of guaiacylpropane- and syringylpropane units. Of these components cellulose is the most desirable since it can be broken down into monomer glucose that can be fermented to ethanol.

However it is not easy to convert lignocellulosic material into sugar. Cellulose fibers are locked into a rigid structure of hemicellulose and lignin. Lignin and hemicelluloses form chemically linked complexes that bind water soluble hemicelluloses into a three dimensional array, cemented together by lignin, that covers cellulose microfibrils and protect them from enzymatic and chemical degradation. These polymers provide plant cell walls with strength and resistance to degradation. This makes lignocellulosic materials a challenge to use as substrates for biofuel production.

A promising route for the conversion of lignocellulose to ethanol is called the enzymatic conversion process. This process consists of five main steps. The first step is the collection and transportation of the biomass to a central process plant. The second step is to pretreat the biomass (prehydrolysis) usually with a unit operation called steam explosion. However, prehydrolysis can be chemical, physical or biological. Diverse techniques have been explored and described for the pretreatment of size-reduced biomass material with the aim of producing substrate that can be more rapidly and efficiently hydrolysed to yield mixtures of fermentable sugars.

These approaches have in common the use of conditions and procedures which greatly increase the surface area to which aqueous reactants and enzymes have access. In particular, the percentage of the major cellulosic materials that are opened up In steam explosion, the biomass is fiberized and the cellulose is fractured making it more susceptible to the third step called enzymatic hydrolysis. Highly specialized enzymes catalyse the depolymerization of the cellulose into glucose. The final two steps are fermentation of the glucose to ethanol and the separation of the ethanol from the aqueous fermentation broth. Ultimately the separation step removes the last remaining water making a water free ethanol suitable for blending with gasoline.

Pretreatments of lignocellulosic biomass, such as steam explosion based pretreatments, generally result in extensive hemicellulose breakdown and, to a certain extent, to the degradation of hemicellulose. This results in the production of soluble and insoluble xylooligosaccharides, acetic acid and furfural. These pretreatment methods may employ hydrolytic techniques using acids (hemicellulose hydrolysis) and alkalis (lignin removal).

A useful form of biomass for the production of ethanol is the agricultural residue, corncobs. It is relatively high in cellulose (35-40% and it is also high in hemicellulose and low in lignin content. The hemicellulose content of corncobs makes up almost 30% of the total dry matter (DM). Moreover, much of the hemicellulose is acetylated which means that breakdown and liquefaction of the hemicellulose leads to the formation of acetic acid. This is a problem, since the acid is a powerful inhibitor of the ethanol fermentation process, remains in the pretreated biomass and carries through to the hydrolysis and fermentation steps. On the other hand the low pH of acetic acid helps in the prehydrolysis process. Hemicellulose is a heteropolymer or matrix polysaccharide present in almost all plant cell walls along with cellulose. While cellulose is crystalline, strong, and resistant to hydrolysis, hemicellulose has a random, amorphous structure with little strength. Hydrolysis of hemicellulose can be relatively easily achieved with acids or enzymes. Hemicellulose contains many different sugar monomers. For instance, besides glucose, hemicellulose can include xylose, mannose, galactose, rhamnose, and arabinose. Xylose is the monomer present in the largest amount.

While cellulose is highly desirable as a starting material for enzymatic ethanol production, high concentrations of the products of enzymatic cellulose and hemicellulose hydrolysis interfere with the performance of cellulose and hemicellulose degrading enzymes. Especially toxic are glucose, cellobiose and xylose, all of which are products of the enzymatic hydrolysis of hemicellulose, and are inhibitors of cellulase enzymes.
A typical cellulose hydrolysis pattern in a batch mode enzymatic process is characterized by a two phase curve, with an initial logarithmic phase followed by an asymptotic phase. During the first phase, cellulose is mainly depolymerised and hydrolyzed into soluble gluco-oligosacharides then cellobiose. Subsequent conversion of cellobiose to glucose is carried out by cellobiases during the second phase of hydrolysis. A rapid release of glucose is normally observed in the initial phase with about half of the cellulose hydrolysed. Hydrolysis of the second half of the cellulose requires days to complete.

Several mechanisms have been proposed for this insufficient hydrolysis phenomenon. However, end-product inhibition of cellulases has been shown to play a major role in hindering continuously fast cellulose to glucose conversion rate.

Several cellulolytic enzymes are involved in the first phase of hydrolysis. The cellobiases are the predominant group of enzymes that carry out the latter step of conversion. As a final product, glucose has a direct inhibitory effect on cellobiase activity.

There is also evidence that glucose has a significant inhibitory impact on exoglucanase and endoglucanase. It has also been shown that cellobiose exhibits a greater inhibitory effect than glucose on cellulase activity during cellulose hydrolysis. It is hypothesized that a high glucose content in the hydrolysate leads to the accumulation of cellobiose which then acts as a secondary inhibitor.

This is a problem since a medium to high-solids operation of the enzymatic hydrolysis of lignocellulose is required to reduce capital costs and increase product concentration to reduce ethanol separation costs.

Enzymatic hydrolysis of lignocellulosic biomass can be carried out in batch or continuous reactors. In a batch process, all components, including pH-controlling substances, are placed in the reactor at the beginning of the hydrolysis. During the hydrolysis process there is no input into or output from the reactor. In a continuous process, there are both input and output flows, but the reaction volume is kept constant.

In an alternative batch process configuration, a fed-batch process, nothing is removed from the reactor during the process, but one substrate component is progressively added in order to control the reaction rate by substrate concentration. The substrate is fed continuously into the reactor over the hydrolysis period without withdrawing any hydrolysate. This type of feeding of the substrates has been found to overcome effects such as substrate inhibition on the product yield.

Of course, substrate inhibition can also be counteracted by increasing the amount of enzyme used in the reaction mixture. However, due to the high cost of enzyme, that approach is uneconomical and the process is normally operated at the lowest enzyme concentration possible.

The main advantages of the fed-batch operation are the possibilities to control the reaction rate by the substrate feed rate. Because practical models for model-based control are rare, fed batch processes are usually run with a predetermined feed profile. Still, it remains a challenge of the enzymatic hydrolysis process to operate the process at the optimal conditions, since the lower the enzyme concentration in the reaction mixture, the higher the danger of substrate or product inhibition of the enzyme.

Usual industrial practice is to develop a reference profile for the substrate feed rate based on operational experience and to implement it in the plant with suitable adjustments to account for the actual conditions in the reactor.

This approach is far from optimal, since it is empirical in nature and operator dependent, which invariably leads to undesired fluctuations in the product yield. Alternatively, mathematical models of the hydrolysis process are used to calculate an optimum substrate flow rate profile off-line and to implement it in the actual fermentation unit to maximize product yield.

A number of different optimization methods and strategies for maximization of the product yield of fed-batch processes were reported. Most of the optimization methods rely on complex mathematical models for computing an optimal feed profile.

Optimal control techniques rely upon an accurate model of the process and for many years mechanistic models have been used to develop optimal control strategies for fed-batch processes. However, mechanistic models of fed-batch processes are usually very difficult to develop due to the complexity and nonlinear nature of the processes.

### SUMMARY OF THE INVENTION

It is now an object of the present invention to provide a process which overcomes at least one of the above disadvantages.

It is a further object to provide a method for the optimization of a fed batch hydrolysis process wherein the process operating parameters are adjusted by means of controlling the feed of the prehydrolysate, preferably the batch volume and/or batch addition frequency of the prehydrolysate and optionally also the enzyme feed, the increase over time in hydrolysate consistency and volume and/or the concentration of sugars released in the reactor, so that the enzymatic hydrolysis is controlled to significantly reduce the impact of cellulase feedback inhibition, especially for low enzyme contents in the reaction mixture, for example enzyme contents lower than 0.5%.

The inventors have now surprisingly discovered that the phenomenon of cellulase product inhibition in the hydrolysate can be reduced, even at very low enzyme loads, by adding the prehydrolysate feed in multiple small batches while closely controlling the batch addition frequency and batch volume, and possibly also the amount of cellulase enzymes, added in each step. In particular, the conditions are chosen such that a high glucose concentration is achieved in the reaction mixture, while the impact of cellulase product and/or substrate inhibition is limited at the same time.

The inventors have discovered that hydrolysis rates in the reaction mixture slow down dramatically as the conversion rate surpasses 70% of the theoretical cellulose to glucose conversion. The inventors have further discovered that the overall time to reach conversion of the total prehydrolysate feed is reduced significantly if the batch addition frequency is equal to one batch each time 70% to 90% conversion of the previous batch is reached in the reaction mixture. The optimum frequency was found to be one batch each time 80% conversion is reached. At an enzyme load of 0.3% in the reaction mixture, the optimum frequency each time 80% conversion was reached was found to be one batch every 105 minutes (min).

In one aspect, the invention provides a process for the hydrolysis of lignocellulosic biomass, such as corncobs, which process includes the steps of filling the reactor with water, adding cellulose enzyme(s) and then carrying out sequential additions of lignocellulosic prehydrolysate feed batches at a preselected batch volume and at a preselected batch addition frequency over a total feed time. Hemicellulolytic enzymes can also be added in steps, either separately or together with the prehydrolysate feed. As the feed is added, the consistency and solids concentration rise until the total desired dry matter content is achieved. The frequency of lignocellulosic prehydrolysate addition is preferably maintained constant over the entire feed time. The batch volume, which means the portion of the total added feed which is added at each feed step, is preferably held constant over the total feed time.

In one aspect, a process for the hydrolysis of lignocellulosic biomass, comprises: filling a reactor vessel with water; adding a cellulase enzyme; and sequentially adding a lignocellulosic prehydrolysate feed into the reactor vessel to produce a reaction mixture, whereby the prehydrolysate feed is added in batches at a preselected batch volume and a batch addition frequency over a total feed time to achieve a preselected final consistency and a preselected dry matter content in a final reaction mixture, the batch addition frequency being equal to one batch each time 70% to 90% of a theoretical cellulose to glucose conversion is reached in the reaction mixture.

In one case, the batch addition frequency is one batch every 80 to 105 min.

In another case, the batch addition frequency is one batch each time 80% of the theoretical cellulose to glucose conversion is reached in the reaction mixture.

In another case, the preselected batch volume and the batch addition frequency are maintained constant throughout the total feed time.

In another case, the preselected batch volume and/or the batch addition frequency are decreased towards an end of the total feed time.

In another case, the batch addition frequency is one batch every 105 min, the preselected consistency is 17% and the preselected addition period is 12 to 35 hours.

In another case, the total feed time is one batch every 17 to 25 hours.

In another case, the total feed time is 20 hours.

In another case, the batch addition frequency is one batch every 105 min, the preselected consistency is 24% and the total feed time is 80 to 120 hours.

In another case, the total feed time is 90 to 110 hours.

In another case, the total feed time is 95 hours.

In another case, the cellulase enzyme is added at an enzyme load of 0.3% in the reaction mixture and the batch frequency is one batch each time 80% conversion is reached.

In another case, the maximum batch addition frequency is one batch every 105 minutes.

In another case, the batch volume is progressively decreased in a second half of the total feed time.

In another case, the batch volume is progressively decreased in a last quarter of the total feed time.

In another case, the enzyme is added in an amount lower than 1% of the final reaction mixture.

### BRIEF DESCRIPTION OF THE DRAWINGS

Other objects and advantages of the invention will become apparent upon reading of the detailed description and upon referring to the drawings in which:

FIGS. 1A and B show feed time profiles used to reach a consistency of 17% DM.

FIG. 1B shows additions of prehydrolysates which were carried out at a frequency of one every 105 min. The lines are not completely straight due to the moisture content of the prehydrolysate.

FIG. 2 shows the change in the conversion time of cellulose to glucose as a function of the feed time of the substrate required to reach 17% consistency.

FIG. 3 shows the change in the conversion time of cellulose to glucose as a function of the feed time of the substrate required to reach 24% consistency. Hydrolysis experiments were carried out at 50° C., pH 5.0. pH adjustment chemical used was liquid ammonia (30%). Commercially available lignocellulolytic enzyme was used at a load of 0.3%. Similar results were obtained at Laboratory (1 kg beaker) and pilot scale (300 kg tank).

FIG. 4 shows an example of 2.5 tonne fed batch hydrolysis of corncobs at 17% followed by a batch ethanologenic fermentation of the resulting hydrolyzate. Hydrolysis was carried out at 50° C., pH 5.0, 0.5% enzyme load. Fermentation was carried out at 33° C., pH 5.3 using an industrial grade C6-fermenting yeast. Hydrolysis and fermentation pH adjustment was carried out using liquid ammonia (30%). Grey circles indicate glucose concentration. Black squares indicate Ethanol concentration;

FIG. 5 shows the results of 17% consistency hydrolysis carried out at 0.3% enzyme load (22CG);

FIG. 6 shows the results of 17% consistency hydrolysis carried out at 0.6% enzyme load;

FIG. 7 shows the impact of combinations of enzyme load and hydrolyzate consistency on conversion time; and

FIG. 8 shows the impact of higher consistency fed-batch hydrolysis on conversion time.

### DETAILED DESCRIPTION OF PREFERRED EMBODIMENTS

Before explaining the present invention in detail, it is to be understood that the invention is not limited to the preferred embodiments contained herein. The invention is capable of other embodiments and of being practiced or carried out in a variety of ways. It is to be understood that the phraseology and terminology employed herein are for the purpose of description and not of limitation.

The invention is directed to ethanol from biomass processes and especially to enzymatic hydrolysis processes. In particular, the invention is directed to processes intended to limit the negative impact of product inhibition in the cellulose containing hydrolysate when lignocellulosic biomass is used as the starting material.

A preferred aspect of the invention is a process for the enzymatic hydrolysis of lignocellulosic biomass for generating a cellulose hydrolysate with reduced feed back inhibition compared to standard fed batch processes. The preferred process of the invention includes the steps of filling the reactor with water and then carrying out sequential additions of lignocellulosic prehydrolysate and enzymes at a constant ratio over a predetermined time. As the prehydrolysate feed and fresh enzymes are added, the consistency and solids concentration rise until the total desired dry matter content is achieved.

A series of enzymatic hydrolysis reactions of a feedstock such as corncobs were conducted at medium and high consistencies that ranged from 17% to 32% to determine optimum process conditions. The effectiveness of each set of hydrolysis conditions was determined by monitoring the time to reach percentages of the theoretical maximum cellulose to glucose conversion in order to evaluate overall cellulose digestibility e.g. t_{90%} the time to reach 90% conversion. The prehydrolysate feedstock was prepared in a batch or continuous steam explosion pretreatment.

Composition analysis was carried out at the analytical laboratory of Paprican (Montreal, Canada), using the TAPPI methods T249 cm-85 and Dairy one (wet chemistry analysis). Total feed times assayed for prehydrolysate and enzyme feeds ranged from 2 hours to 140 hours (h)

The hydrolysis process operating conditions were screened with respect to high cellulose to glucose conversion rates obtained at low enzyme loading. The hydrolysis conditions were chosen to ensure a high glucose concentration was achieved, while the impact of product inhibition of the cellulases was limited at the same time.

Hydrolysis time of the corncobs prehydrolysate at 17% consistency was generally less than 100 hours.

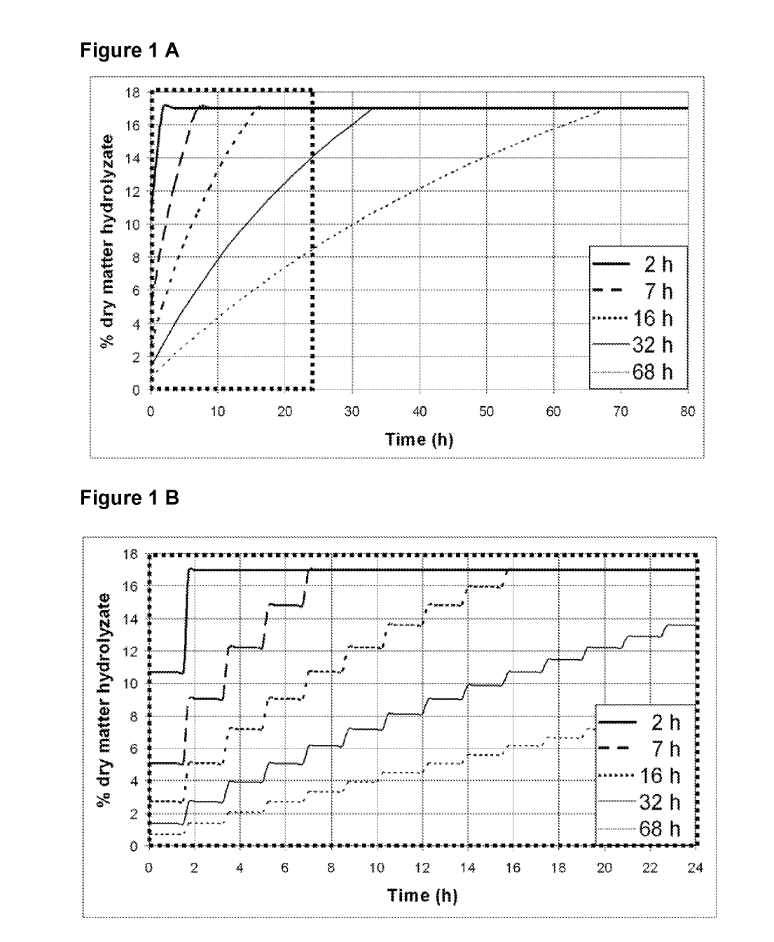

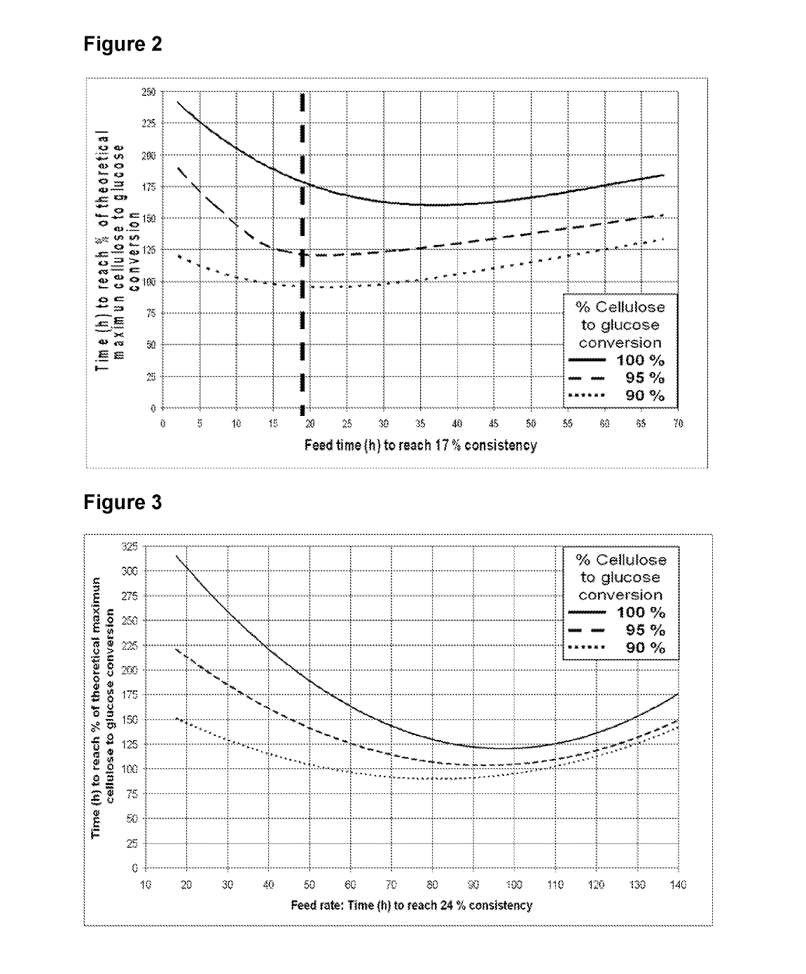

Quantification of soluble products from pretreatment and enzymatic hydrolysis was carried out by HPLC analysis. Target molecules were monitored to determine the relative contents of cellulose and downstream inhibitors in the prehydrolysate obtained. The target molecules were sugar monomers such as glucose and xylose. The summary results of the test treatment series are plotted in FIGS. 1 and 2.

As shown in FIGS. 1A and B, in a fed batch hydrolysis the feed and enzymes can be added in different ways. We have previously found that small sequential additions of new feed and enzymes carried out on a regular basis gave much faster hydrolysis than adding the total mass of feed and enzymes in one addition. In each case, a predetermined amount of water was added to a beaker and then the feed and enzymes were added over different feed time periods that ranged from 2 h to 68 h. As mentioned above, the enzyme feed can be correlated with the prehydrolysate feed, or carried out completely independently. The complete enzyme charge can also be added in one single feed step at the beginning of the feed time. All hydrolysates were continuously maintained at the same enzyme load using sequential additions of enzyme.

FIG. 2 shows that adding the prehydrolysate and enzyme over a period of 18 hours to reach 17% consistency led to the shortest time to reach up to 90% or 95% conversion. To achieve 100% conversion, the feed time should be extended to about 40 h. The overall hydrolysis time almost doubles between 90% and 100% conversion. Similar results were obtained in the lab (1 kg beaker) and at pilot scale (300 kg tank) using 18 h feed time. Additions of prehydrolysate were carried out each 105 min. This number was chosen based on our experience that it requires about 105 min for liquefaction of the cellulose to occur. Acceptable feed frequencies would be one every 80 min to one every 105 min. In each case, substrate was added at intervals of 105 min. The batch volume, which means the quantity of substrate added at each additional step was varied to give the desired consistency in the desired total feed time.

FIG. 3 shows the change in the conversion time of cellulose to glucose as a function of the total feed time of the substrate to reach 24% consistency.

The optimum total feed time to reach 80%, 85% or 90% conversion of 24% consistency hydrolysate was 80 h, 90 h and 100 h respectively. At 24% consistency 150 grams per liter (g/L) glucose were detected after 180 h. Similar results were obtained in the lab (1 kg beaker) and at pilot scale (300 kg tank) using 140 h total feed time. In each case the substrate was added at intervals of 105 min. The batch volume, which means the quantity of substrate added was varied to give the desired consistency in the desired total feed time.

Acceptable conditions for fed batch hydrolysis of corncobs were found to be a 12 h to 35 h total feed time for 17% consistency hydrolysis or 80 h to 120 h total feed time for 24% consistency hydrolysis. Improved results were achieved using a total feed time of 17 h to 25 h at 17% consistency or 90 h to 110 h total feed time at 24% consistency. Optimal results were achieved using 25 h or 95 h total feed time at 17% or 24% consistency, respectively.

The governing factors for the effectiveness of fed batch hydrolysis were found to be total feed time and batch addition frequency.

The enzyme used was a commercial product from Novozymes. Novozym 22CG is a liquid product (17% DM, 10.5%, w/w, protein on a DM basis) supplied in 25 kg pails at a price of $US 21.8 per kg on a DM basis. The enzyme load was measured as a ratio, expressed as a %-value, of the desired/target total amount of biomass that ends up being present in the hydrolysis tank when the feed process is complete. Weights of both the enzyme and the biomass feed are expressed on a dry matter basis. For instance, 3 kg of 10% DM enzyme solution (i.e.0.3 kg on a DM basis) would be initially added to some water (i.e. 488 liters initially added) when the hydrolysis tank was to be fed with a total of 200 kg of 50% DM biomass (i.e. 100 kg DM) over the entire feed process. In that instance, the final consistency of the hydrolysis would have been 17% (rounded), since a total of 100 kg of biomass on a DM basis would have been added to a total of 588 Liters composed of the initial 488 liters of water plus the 100 kg of water present in the 200 kg of 50% DM biomass fed into the hydrolysis tank.

Hydrolysis experiments were carried out in 1 kg-beakers or in 300 kg-tanks of an indoor pilot plant. All hydrolysis experiments were carried out in fed-batch mode.

Fed-batch hydrolysis is carried out by filling a tank with water and then adding quantities of feed and enzymes in a constant ratio over a predetermined time. As the feed and enzymes are added, the consistency and solids concentration rise until the total desired dry matter content is achieved. Biomass consistencies were adjusted to various levels from 17% to 28%. Enzyme loads ranged from 0.16% to 1.44% (w/w, DM raw cob) of 22CG enzyme. Co-addition of prehydrolyzate and 22CG liquid enzyme was made over periods that ranged from 2 hours to 140 hours. Sequential additions were carried out at intervals of 105 min in between each addition. The first addition of prehydrolyzate and enzyme were carried out at time zero of the hydrolysis feed time. Hydrolysis experiments were carried out at a temperature of 50 oC and pH 5.0. These values were previously determined as 22CG optima.

The progress of each hydrolysis was assessed daily. The experiments were monitored until no more significant increase in glucose concentration was detected. Dry matter content was measured by drying solid (1 g to 2 g) and liquid (5 g to 10 g) samples at 130 oC for a period of 16 to 24 hours.

Cellulose to glucose conversion is expressed as a percentage of the maximum theoretical conversion of cellulose to glucose. Hydrolysis time to reach 90% of the maximum theoretical cellulose to glucose conversion (t 90%) was used as indicator of hydrolysis efficiency.

A series of fed-batch hydrolysis experiments were carried out at 17% and 24% consistency to assess the impact of enzyme load on cellulose to glucose conversion time of washed pretreated cob.

Fed-batch hydrolysis experiments were carried out by adding quantities of feed and enzymes in a constant ratio over a predetermined time. This time is called the feed time and is generally shorter than the hydrolysis time. Ten additions of washed pretreated cob were carried out over 16 hours to reach 17% consistency. It took 140 hours to carry out 80 additions of cob and enzymes to reach 24% consistency. Enzyme loads were varied between 0.16% to 1.44% (w/w, DM, raw cobs) during the 17% consistency hydrolysis experiments.

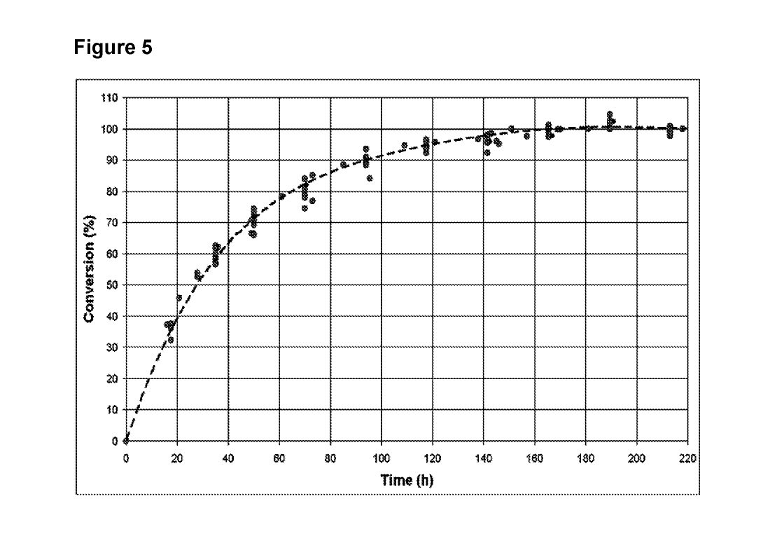

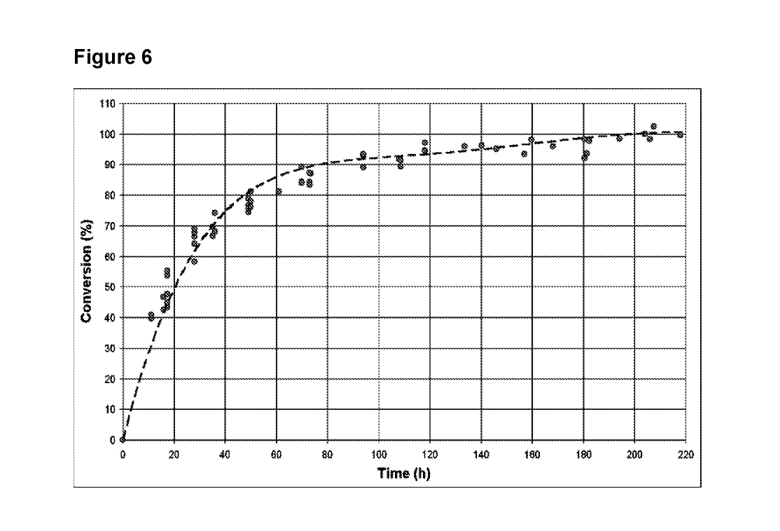

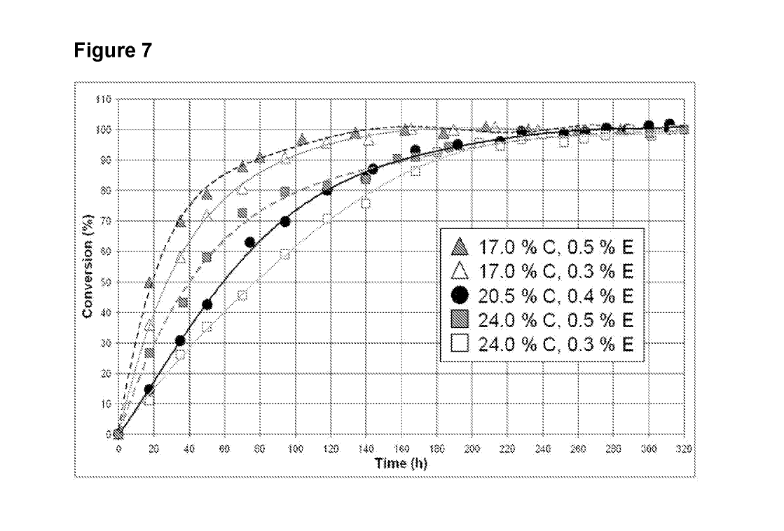

FIGS. 5 and 6 show the conversion of cellulose to glucose over time at 17% consistency and 0.3% and 0.6% load of enzyme, respectively. It is apparent that the conversion rate was virtually independent on enzyme load. Complete cellulose to glucose conversion was achieved using enzyme loads that ranged from 0.3% to 1.4%. t 90% ranged from 36 hours to 96 hours depending on the enzyme load.

A series of fed-batch hydrolysis experiments were carried out at 17% and 24% consistency to evaluate the impact of feed time on cellulose to glucose conversion time of washed pretreated cob. Feed time is the total time over which the biomass and enzyme are added to the hydrolysis tank. Fed-batch hydrolysis experiments were carried out by adding quantities of feed and enzymes in a constant ratio over a predetermined time. The substrate addition intervals (batch addition intervals) were maintained constant throughout. The quantity of washed pretreated cob added at one time (batch volume) was varied to give the desired consistency in the desired feed time. Shorter feed times tended to negatively affect overall hydrolysis conversion time. This negative impact on cellulose to glucose conversion of adding most biomass during the early phase of the hydrolysis was found to be more significant at higher consistency.

At 17% consistency, the shortest t 90% (90 hours) was achieved with a 20 h-feed time. At a fed time of 16 hours to reach a consistency of 17% the t 90% was 96 hours, using 0.3% load of enzyme.

A matrix of experiment was carried out to investigate the relationship between enzyme load and percentage consistency of cob hydrolysate. Table I summarizes the two level factorial design with center point matrix of experiments used to determine conditions of pilot scale (250 kg) fed-batch hydrolysis and Table II shows the results achieved. The dependent variable was t 90%. The range of enzyme load used was 0.3% to 0.5%. The range of hydrolyzate consistency assayed was 17% to 24%.

TABLE I
Matrix of experiment

| Two level factorial | Consistency (%) | | |
|---|---|---|---|
| design with center point | 17.0 | 20.5 | 24 |

| | | | | |
|---|---|---|---|---|
| Enzyme | 0.5 | ✓ | | ✓ |
| load | 0.4 | | ✓ | |
| (%, w/w, DM) | 0.3 | ✓ | | ✓ |

TABLE II
Results of experiment

| | Consistency (%) | | |
|---|---|---|---|
| t_{90%} (h) | 17.0 | 20.5 | 24 |

| | | | | |
|---|---|---|---|---|
| Enzyme | 0.5 | 78 | | 160 |
| load | 0.4 | | 156 | |
| (%, w/w, DM) | 0.3 | 96 | | 180 |

Replicate experiments showed that the variability in t_{90%} values was equal to +/−5 h.

Table II shows that the time to reach 90% conversion of 24% consistency hydrolyzate is about two times longer than at 17% consistency although the ratio of enzyme and biomass remains the same on a dry matter basis. Similar results were obtained at 0.3% and 0.5% load of enzyme. These results surprisingly indicated that the increase in conversion time associated with higher consistency hydrolysis is substantially independent of the ratio of enzyme used. The value of t 90% observed for the central point of the matrix (Table II) confirms that both variables (enzyme load and hydrolyzate consistency) are independent i.e. lack of symmetry in variance.

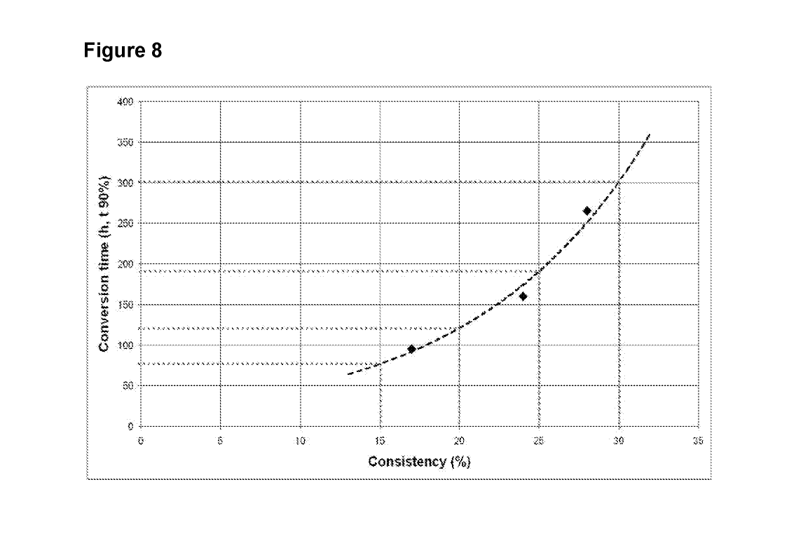

FIG. 8 shows the impact of higher consistency fed-batch hydrolysis on conversion time. Fed-batch hydrolysis experiments were carried out using the similar ratio of enzyme and biomass on a dry matter basis (0.5%) and consistencies that range from 17% to 28% (black diamond). The dashed line shows that the impact of higher consistency on conversion time was not linear but exponential although the ratio of enzyme and biomass was maintained constant. A correlation coefficient (R2) of 0.98 was obtained. Dotted grey lines in FIG. 8 show that each increase of 5% consistency between 15% and 30% consistency does not lead to the same increase in conversion time. Increases of 5% consistency between 15% and 20% consistency led to 45 hours increase in conversion time while between 20-25% and between 25%-30% the increases were 70 hours and 110 hours respectively.

The results of the experiments showed that the minimum load of enzyme needed to reach complete cellulose to glucose conversion at 17% consistency was between 0.2% and 0.3%. A load of 0.3% resulted in a t 90% of 95 hours at 17% consistency and 178 hours at 24% consistency. It would take almost five times more enzyme to reach 90% conversion in the same time at 24% consistency than at 17%. The feed rate profile shows that the conversion time of 24% consistency hydrolyzate can be significantly reduced by selecting appropriate feed times. A feed time of 100 hours instead of 140 hours decreased the t 90% value of 24% consistency from 178 hours to 120 h.

These results also showed that a feed time of 16 hours used to carry out 17% consistency hydrolysis was very close to the optimum feed time. A feed time of 20 hours instead of 16 hours would lead to a slight decrease in t 90% of 6 hours i.e. from 96 h to 90 h. The conversion time observed with sequential addition of biomass only or with biomass and enzyme co-addition were similar. It took 100 hours to reach 90% conversion with sequential addition of biomass only.

The results also confirmed that addition of all or most of the biomass at the very beginning of the hydrolysis led to a significantly longer conversion time. The impact of higher consistency on conversion time was not linear but exponential between 15% and 30% consistency, although the ratio of enzyme to biomass was maintained constant.

Surprisingly, the increase in conversion time associated with higher consistency hydrolysis was independent of the enzyme load. The difference in conversion rates resulting from the use of different enzyme loads was not dependent on hydrolyzate consistency.

### EXAMPLE

Ground corncobs of 0.5 to 1 cm³ particle size were pretreated by autohydrolysis steam explosion pretreatment at 205 oC, i.e. cooking pressure of 235 psig for a residence time of 8 min. The cooked corncobs were then washed and pressed to remove soluble xylooligosaccharides and toxins prior to enzymatic hydrolysis. The washed and pressed cake of prehydrolysed corncobs was shredded in a garden shredder and then diluted with fresh water to the desired consistency for hydrolysis and fermentation.

A 2.5 ton hydrolysis and fermentation trial was carried out at 17% consistency. Enzymatic hydrolysis was carried out at 50° C., pH 5.0. Fermentation was carried out at 33° C., pH 5.3. Aqueous ammonia at 30% concentration was used to adjust pH. Commercially available lignocellulosic enzyme product (Novozym 22CG) and industrial grade ethanologenic yeast were used.

Pilot scale hydrolysis and fermentation was carried out in a heat traced, jacketed 6000 liter tank equipped with a recirculation pump, a high speed mixer and a wiper.

Co-addition of corncobs prehydrolysate at 35% DM and liquid enzyme was made over a period of 16 h. Ten additions were carried out with a gap of 105 min in-between each addition such as described in FIGS. 1A and 1B (dotted line). The first addition of prehydrolysate and enzyme was carried out at time zero of the hydrolysis feed time. This feeding procedure was determined as being in the range of optimum feed time to reach 90% to 95% of the maximum theoretical cellulose to glucose conversion of 17% consistency pretreated corncobs hydrolyzate at laboratory and smaller pilot scale (FIG. 2).

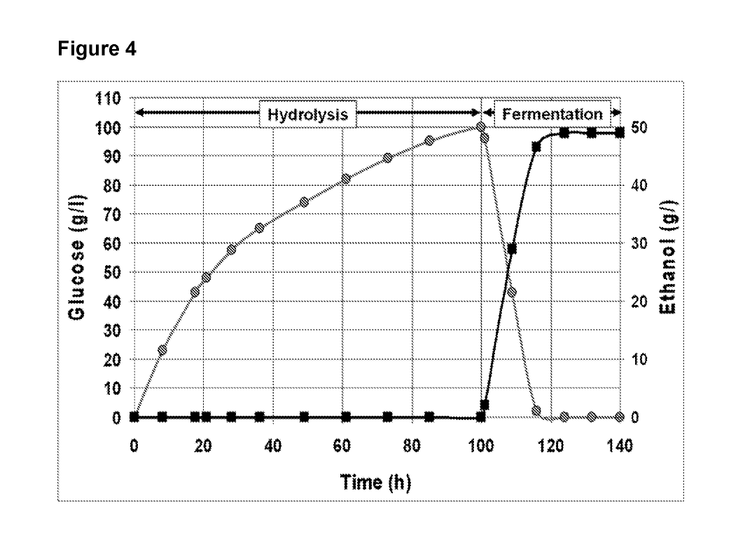

Results of the pilot scale trial showed that a concentration of 100 g/L glucose was reached at t_{90%} i.e. 100 h hydrolysis (FIG. 4). Hydrolysis time of the 2.5 tonnes trial was in accordance with above discussed results obtained at laboratory and 300 kg pilot scale.

In this example a titer of 5% alcohol was reached by 20 hours fermentation.

What is claimed is:

1. A process for the hydrolysis of a lignocellulosic biomass, comprising:
pretreating the biomass by grinding the biomass to 0.5 to 1 cm³ particle size and subjecting the ground biomass to autohydrolysis by steam explosion to produce a prehydrolysate feed,
filling a reactor vessel with a volume of water;
adding a cellulase enzyme to the volume of water in the reactor vessel; and
sequentially adding an amount of the prehydrolysate feed into the reactor vessel to produce a reaction mixture, said amount of prehydrolysate feed being added in multiple sequential batches at a preselected batch volume and batch addition frequency over a total feed time, the batch addition frequency being equal to one batch each time 80% of a theoretical cellulose to glucose conversion of the preceding batch is reached in the reaction mixture, wherein optionally further cellulase enzyme is added to said reactor vessel with each said batch,
wherein for maximizing cellulose to glucose conversion and reducing the total enzyme load of all cellulase enzyme added to the reactor vessel for the amount of prehydrolysate feed to less than 1% w/w dm of the amount of prehydrolysate feed, the batch volume and feed time are selected to achieve a consistency of 17-24% in the reactor vessel at a corresponding feed time of 12-120 hours, the batch addition frequency is one batch every 80 to 105 min, the batch addition frequency being independent of the consistency, and the preselected batch volume and batch addition frequency are maintained constant throughout the total feed time.

2. The process of claim 1, wherein the batch addition frequency is one batch every 105 min, and the total feed time is 12 to 35 hours.

3. The process of claim 2, wherein the total feed time is one batch every 17 to 25 hours.

4. The process of claim 3, wherein the total feed time is 20 hours.

5. The process of claim 1, wherein the lignocellulosic biomass is corncob.

6. The process of claim 1, wherein the consistency is about 17%, the batch addition frequency is one batch every 105 min, the total feed time is 12-35 hours and the total enzyme load is 0.3% w/w dm.

7. The process of claim 6, wherein the batch addition frequency is one batch every 105 min, the total feed time is 25 hours and the total enzyme load is 0.3% w/w dm.

8. The process of claim 1, wherein the consistency is about 24%, the batch addition frequency is one batch every 80 min, the total feed time is 80-120 hours and the total enzyme load is 0.3% w/w dm.

9. The process of claim 8, wherein the consistency is about 24%, the batch addition frequency is one batch every 80 min, the total feed time is 95 hours and the total enzyme load is 0.3% w/w dm.
